(19) United States
(12) Patent Application Publication
(10) Pub. No.: US 2025/0235326 A1
(43) Pub. Date: Jul. 24, 2025

(54) ORTHOPEDIC ALIGNMENT DEVICE

(71) Applicant: Arthrosurface Incorporated, Franklin, MA (US)

(72) Inventor: Steven W. EK, Bolton, MA (US)

(73) Assignee: Arthrosurface Incorporated, Franklin, MA (US)

(21) Appl. No.: 19/172,366

(22) Filed: Apr. 7, 2025

Related U.S. Application Data
(63) Continuation of application No. PCT/US23/76479, filed on Oct. 10, 2023.
(60) Provisional application No. 63/378,779, filed on Oct. 7, 2022.
(60) Provisional application No. 63/481,089, filed on Jan. 23, 2023.

(51) Int. Cl. A61F 2/46

(52) U.S. Cl. CPC A61F 2/46; A61F 2002/4687

(57) ABSTRACT

Described herein are pin guide devices and related systems, methods, and kits useful in the formation of a glenoid implant site. The pin guide device includes a cup comprising an inner concave surface, an outer convex surface, a cup wall between the inner and outer surfaces, and a substantially circular base, an elongated opening extending through the cup wall, an elongated opening extending through the cup wall, an elongated opening extending through the cup wall, and a stem comprising a passageway for receiving the pin, the stem extending through the elongated opening and slidable within the elongated opening.

### CROSS-REFERENCE

This application claims the benefit of and priority to U.S. Patent Application No. 63/378,779, titled “Orthopedic Alignment Device”, and filed Oct. 7, 2022, and U.S. Patent Application No. 63/481,089, titled “Orthopedic Alignment Device”, and filed Jan. 23, 2023, each of which is incorporated herein by reference in its entirety.

### TECHNICAL FIELD

The present disclosure is related to devices, systems, methods, and kits for repair of defects that occur in articular cartilage covering the surface of bones. More particularly, for the guiding of surgical guide pins into a bone for implant surgery.

### BACKGROUND

Articular bones in the human body have articular cartilage covering the ends of the bone, particularly where one bone interfaces with another bone such as in a joint. Articular cartilage is smooth, load bearing, and lubricious allowing one bone to slip past another bone while maintaining strength during movement. When a bone is injured, this articular cartilage may be damaged. Furthermore, as the body ages articular cartilage can naturally break down causing bone to rub on bone leading to pain for the patient, reduced mobility, and osteoarthritis.

In some cases, the extent of damage necessitates repair to the cartilage using one or more implants. Guiding an implant or an anchor for an implant into bone at the proper angle is important to ensure the implant is stable, allows for mobility of the joint and maintains long-term load bearing of the implant for the patient. Many surgeons use a pin guide device that is placed over the surgical implant location and includes an open passageway that is oriented so that the longitudinal axis is aligned with the proper location and angle for the implant so a pin can be inserted through the passageway and into the bone as a guide for subsequent implant insertion. For example, this technique can be used in the glenoid bone of the scapula when placing a humeral implant.

Previously, guide devices were designed based on a scan of the patient's anatomy and bone structure to create a custom guide device configuration to mate with each contour of the of the patient's bone. However, creating a custom guide device is costly and can inhibit the surgeon from making small adjustments to the angle of pin insertion into the bone during surgery based on what the surgeon finds during surgery.

For example, during surgery a surgeon may identify the alignment of the pin in the guide device may be centered on native bone that has poor bone quality, an issue with soft tissue, or an osteocyte, all factors that may not be typically identified during preoperative scans and would necessitate moving the implant location or angle of guide pin insertion. For example, if the implant was placed in a part of the bone with poor bone quality, then the implant may separate from the bone resulting in the need for subsequent revision surgery and reduced patient mobility.

Embodiments described herein are comprised of building blocks that can be taken off the shelf and configured to an individual patient. Using off the shelf components reduces the cost of custom manufactured guide devices while still allowing the proper size to be selected for and individual patients' anatomy. Furthermore, the described embodiments may have two axes of rotation which allows the angle and location for the implant to be adjusted during surgery based on the bone anatomy of an individual patient.

### SUMMARY

In one aspect, embodiments described herein relate to pin guide devices useful in the formation of a glenoid implant site and/or placement of a glenoid implant. In another aspect, embodiments described herein relate to systems and/or kits that include pin guide devices useful in the formation of a glenoid implant site and/or placement of a glenoid implant. In yet another aspect, embodiments described herein relate to methods of forming a glenoid implant site and/or placing a glenoid implant using pin guide devices described herein.

In some embodiments, a pin guide device is described that comprises a cup comprising an inner concave surface, an outer convex surface, a cup wall between the inner and outer surfaces, and a substantially circular base, an elongated opening extending through the cup wall, an elongated opening extending through the cup wall, an elongated opening extending through the cup wall, and a stem comprising a passageway for receiving the pin, the stem extending through the elongated opening and slidable within the elongated opening. In some embodiments, the stem protrudes from a pin receiving member comprising a rounded surface configured to contact the inner concave surface of the cup. In some embodiments, the pin receiving member is configured to be locked at a desired location within the elongated opening. In some embodiments, the cup comprises one or more first mating members, and the pin receiving member comprises one or more second mating members, wherein the first and second mating members are configured to interact with each other to hold the stem at a desired location within the elongated opening. In some embodiments, the cup wall comprises a mating surface configured to receive a guide device placement tool. In some embodiments, one or more first mating members are positioned on the outer convex surface of the cup. In some embodiments, the mating surface is an annular indentation. In some embodiments, the device further comprises one or more angle markings on the outer convex surface of the cup corresponding to an offset angle relative to the base. In some embodiments, the device comprises an axis marking on the stem, the axis marking being parallel to a longitudinal axis of the stem and configured to be aligned with the one or more angle markings. In some embodiments, the base is separable from the cup. In some embodiments, the cup comprises a first alignment feature and the base comprises a second alignment feature, the first and second alignment features being connectable. In some embodiments, the cup wall comprises a mating surface configured to receive a guide device placement tool. In some embodiments, the device further comprises at least one protrusion extending from a bone facing surface of the base. In some embodiments, the at least one protrusion comprises a first protrusion, a second protrusion, and a third protrusion. In some embodiments, the device one of the first, second, and third protrusions is longer than the other protrusions. In some embodiments, further comprising a first bridge extending from the stem to a first end offset from the longitudinal axis of the stem, a second bridge extending from the stem to a second end offset from the longitudinal axis of the stem, and an implant sizer connecting the first end to the second end. In some embodiments, the device further comprises a first bridge extending from the stem to a first end offset from the longitudinal axis of the stem, a second bridge extending from the stem to a second end offset from the longitudinal axis of the stem, and an implant sizer connecting the first end to the second end. In some embodiments, the device further comprises a first bridge extending to a first distal end perpendicular to the longitudinal axis of the stem, a second bridge extending to a second distal end perpendicular to the longitudinal axis of the stem, and an implant sizer connecting the first distal end to the second distal end. In some embodiments, the implant sizer is circular or semi-circular.

In some embodiments, a method of forming a glenoid implant site in the glenoid of a patient in need of a glenoid implant is described, the method comprising the steps of: providing a pin guide device kit comprising a plurality of pin guide devices that are non-specific to the patient, selecting a pin guide device from the plurality of pin guide devices based upon an anatomy of the patient, locking a position of a pin receiving member within the selected pin guide device based upon an anatomy of the patient, and advancing a pin through the pin receiving member and into the glenoid of the patient. In some embodiments, the method further comprises the steps of advancing an annulated drill over the pin to form a pilot hole in the glenoid of the patient and advancing an anchor over the pin and into the pilot hole. In some embodiments, each pin guide device of the plurality of pin guide devices comprises a cup comprising an inner concave surface, an outer convex surface, a cup wall between the inner and outer surfaces, and a substantially circular base; and an elongated opening extending through the cup wall. In some embodiments, the pin receiving member comprises a rounded surface configured to contact the inner concave surface of the cup, and a stem protruding from the pin receiving member comprising a passageway for receiving the pin, the stem extending through the elongated opening and slidable within the elongated opening. In some embodiments, the method further comprises the steps of securing a tray to the anchor and coupling the tray with an implant. In some embodiments, the method each pin guide device of the plurality of pin guide devices comprises a cup comprising an inner concave surface, an outer convex surface, a cup wall between the inner and outer surfaces, a substantially circular base, and an elongated opening extending through the cup wall. In some embodiments, of the method the pin receiving member comprises a rounded surface configured to contact the inner concave surface of the cup, and a stem protruding from the pin receiving member comprising a passageway for receiving the pin, the stem extending through the elongated opening and slidable within the elongated opening.

In some embodiments, a system for guiding a surgical pin into bone is described, the system comprising: a pin guide device comprising a cup comprising an inner concave surface, an outer convex surface, a cup wall between the inner and outer surfaces, a substantially circular base, and an elongated opening extending through the cup wall; a stem comprising a passageway for receiving the pin, the stem extending through the elongated opening and slidable within the elongated opening, and a guide device placement tool. In some embodiments, of the system the cup wall comprises a mating surface configured to receive the guide device placement tool. In some embodiments, of the system the mating surface is an annular indentation.

### BRIEF DESCRIPTION OF THE DRAWINGS

FIG. 1 shows components of an alignment system according to embodiments described herein.

FIG. 2 shows a closeup of cups components according to embodiments described herein.

FIG. 3 shows the underside of a cup and a pin receiving member according to embodiments described herein.

FIG. 4 shows a mated cup and pin receiving member according to embodiments described herein.

FIG. 5 shows a pin guide device placed over an implant location and a surgical guide pin inserted into a pin receiving member according to embodiments described herein.

FIG. 6 shows a pin guide device placed over an implant location and a surgical guide pin inserted into a pin receiving member according to embodiments described herein.

FIG. 7 shows base comprising protrusions according to embodiments described herein.

FIG. 8 shows a closeup of a pin guide device with offset angle markings according to embodiments described herein.

FIG. 9 shows a mated cup and pin receiving member with offset angle markings according to embodiments described herein.

FIGS. 10A and FIG. 10B show different angles of a mated cup, a pin receiving member, and a base with protrusions and offset angle markings according to embodiments described herein.

FIGS. 11A and FIG. 11B show different angles of a surgical guide pin embedded in the glenoid region of the scapula bone according to embodiments described herein.

FIGS. 12A-12D show various views of a base according to embodiments described herein. FIG. 12A illustrates a side view of a base according to embodiments described herein. FIG. 12B illustrates a perspective side view of 12A according to embodiments described herein. FIG. 12C illustrates a bottom view of 12A. FIG. 12D illustrates an angled top and side view of 12A according to embodiments described herein.

FIGS. 13A and 13B show a base and a guide device placement tool according to embodiments described herein. FIG. 13A illustrates an exploded view of a base and a guide device placement tool according to embodiments described herein. FIG. 13B illustrates a coupled view of a base and a guide device placement tool according to embodiments described herein.

FIG. 14 shows a base coupled with a guide device placement tool placed over a glenoid according to embodiments described herein.

FIGS. 15A-15C show various views of a cup according to embodiments described herein. FIG. 15A illustrates an angled top view of a cup according to embodiments described herein. FIG. 15B illustrates a top-down view of 15A. FIG. 15C illustrates a side view of FIG. 15A according to embodiments described herein.

FIGS. 16A-16E show various views a stem according to embodiments described herein. FIG. 16A illustrates a first side view of a stem according to embodiments described herein. FIG. 16B illustrates a second side view of FIG. 16A according to embodiments described herein. FIG. 16C illustrates a top view of FIG. 16A according to embodiments described herein. FIG. 16D illustrates an angled top and side view of FIG. 16A according to embodiments described herein. FIG. 16E illustrates an angled bottom side view of FIG. 16A according to embodiments described herein.

FIGS. 17A and 17B illustrate an exploded and compact view of a pin guide alignment system according to embodiments described herein. FIG. 17A illustrates an exploded view of a pin guide alignment system with a stem coupled to cup and a guide placement tool coupled to a base and placed over a glenoid region of a scapula according to embodiments described herein. FIG. 17B illustrates a compact view of a pin guide alignment system with a stem coupled to cup coupled to a base and placed over a glenoid region of a scapula by a guide placement tool according to embodiments described herein.

FIGS. 18A-18D illustrate various views of an implant sizer according to embodiments described herein. FIG. 18A illustrates a top side angled view of an implant sizer according to embodiments described herein. FIG. 18B illustrates a bottom view of FIG. 18A according to embodiments described herein. FIG. 18C illustrates a long side view of FIG. 18A according to embodiments described herein. FIG. 18D illustrates a short side view of FIG. 18A.

FIGS. 19A and 19B illustrate an exploded and compact view of a pin guide alignment system according to embodiments described herein. FIG. 19A illustrates an exploded view of a pin guide alignment system with an implant sizer and coupled to a base and placed over a glenoid region of a scapula with a guide placement tool according to embodiments described herein. FIG. 19B illustrates a compact view of a pin guide alignment system with an implant sizer coupled to a stem coupled to cup coupled to a base and placed over a glenoid region of a scapula by a guide placement tool according to embodiments described herein.

FIG. 20 illustrates a compact view of a pin guide alignment system with a surgical guide pin disposed through an implant sizer coupled to a stem coupled to cup coupled to a base and placed over a glenoid region of a scapula by a guide placement tool according to embodiments described herein.

FIG. 21 illustrates a surgical guide pin disposed in a scapula bone according to embodiments described herein.

FIG. 22 illustrates an embodiment of a pin guide alignment system placed over a glenoid region of a scapula according to embodiments described herein.

FIGS. 23A-23C illustrate several views of an embodiment of a pin guide alignment system and an implant sizer placed over a glenoid region of a scapula according to embodiments described herein.

FIG. 24 illustrates an embodiment of a surgical guide pin which has been placed within the glenoid region of a scapula bone according to embodiments described herein.

### DETAILED DESCRIPTION

This disclosure presents various systems, components, and methods related to a pin guide device. Each of the systems, components, and methods disclosed herein provides one are more advantages over traditional systems, components, and methods. Various embodiments of the pin guide devices, methods, and systems are disclosed herein.

As used herein, the terms “about” or “substantially” mean within ±10% of the value it modifies. For example, “about 1” means “0.9 to 1.1”, “about 2%” means ∂1.8% to 2.2% “, “about 2% to 3%” means ∂1.8% to 3.3%”, and “about 3% to about 4%” means “2.7% to 4.4%.” Unless otherwise clear from the context, all numerical values provided herein are modified by the term “about”.

As a predicate to the placement of a glenoid implant in embodiments of the present invention, an understanding of a patient's anatomy may be utilized for the purposes of pre-operative planning. For example, a three-dimensional (3D) model may be constructed based upon any known imaging modality, such as magnetic resonance imaging (MRI) or a computed tomography (CT) scan performed on an individual patient to map a patient's specific bone configuration and topography. In certain other embodiments, a 3D model of a patient's anatomy may be constructed based upon a series of x-rays or other imaging modalities that are compared to a database or other similar catalog of bone models for a population of patients in order to estimate the anatomy of a specific patient. These 3D models may be used to determine the ideal angle and location to place a surgical guide pin for subsequent implant placement into a patient's bone. It is important that the guide pin be placed at the proper location and angle for subsequent proper implant placement. In some embodiments, this implantation site is the glenoid portion of a scapula. In some embodiments, the bone model is used to configure the pin guide device so that the location and angle for surgical guide pin insertion is optimized for the patient's anatomy.

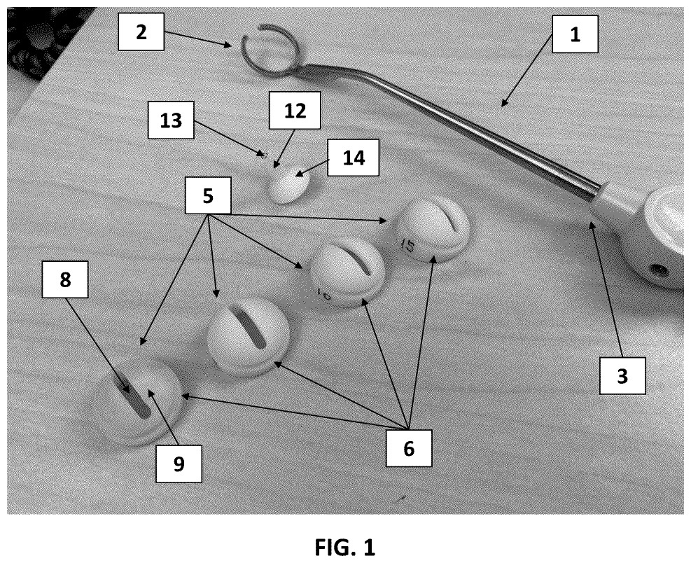

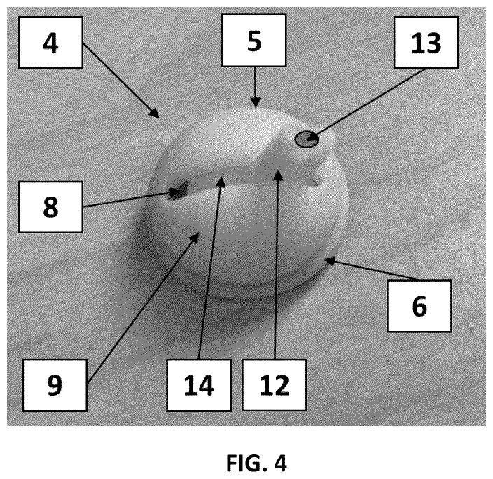

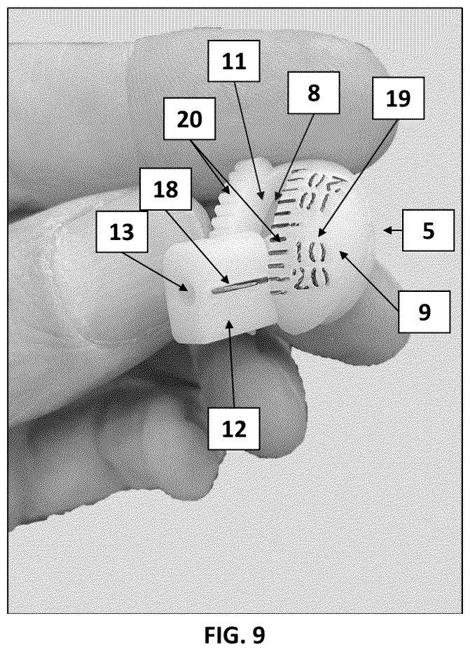

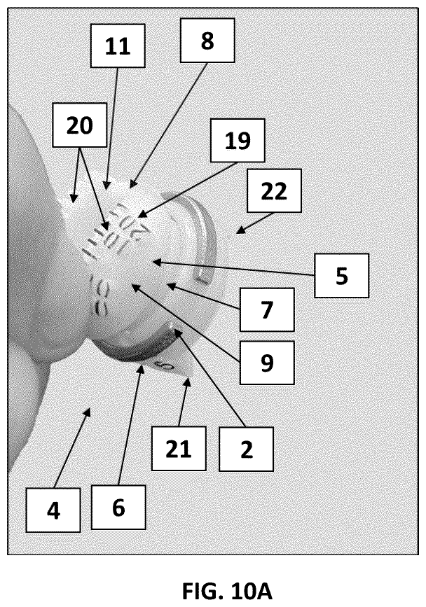

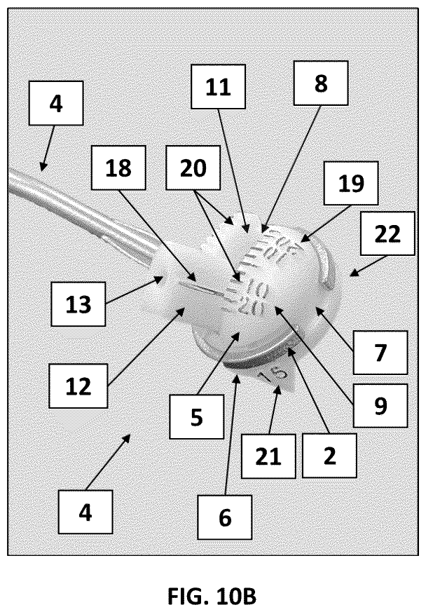

FIGS. 1-10 show embodiments of devices used in a system for proper placement of a surgical pin into bone for subsequent placement of an anchor and/or other components of a glenoid implant. The system may include a guide device placement tool 1 comprising an arm 2 and a handle 3. The handle 3 may be used to hold the tool 1 and maneuver it to grasp a pin guide device 4, which generally comprises a cup 5 and a pin receiving member 14 (as shown assembled together in FIG. 4). In some embodiments, the arm 2 may be shaped with two curved hands with a substantially circular opening between the hands. In some embodiments, the hands interact with an annular indent 7 in the cup portion of the pin guide device 4 to grasp cup 5.

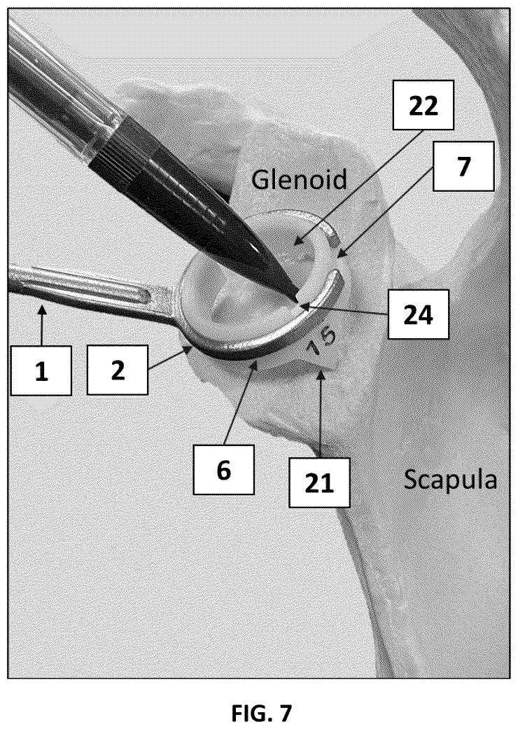

The cup 5 of the pin guide device 4 may comprise a circular base 6 and a convex dome extending from the base 6. In some embodiments, the base 6 further comprises tapered protrusions, as described with reference to FIG. 7. In some embodiments, a cup further comprises an elongated opening 8 extending through the cup wall 11. The pin guide device 14 may also comprise a pin receiving member comprising a stem 12 with a passageway 13 therethrough. In some embodiments, the stem further comprises a stem longitudinal axis marker 18.

As shown in FIG. 1, the pin guide device 4 may be provided with the cup 5 and pin receiving member 14 as separate components, which are fit together for use, as shown in FIG. 4. In other embodiments, the pin guide device 4 may be provided in a kit with the cup 5 and pin receiving member 14 as an integrated assembly. In either case, the pin guide device 4 is assembled prior to use such that the cup base 6 may come into contact with bone.

In some embodiments, the passageway 13 in the stem 12 of pin guide device 14 is configured to receive a surgical guide pin. In some embodiments, the cup 5 of the pin guide device 4 fits around or within an implant site such that the cup 6 is placed against bone at the implant site. After this contact is made, the cup 5 may be rotated and the stem 12 of pin receiving member 14 may be manipulated within an elongated opening 8 within the cup 5 to provide a proper orientation for insertion of the surgical guide pin through the passageway 13 of the stem 12 and into bone. After the surgical guide pin 23 is placed in the bone of the patient, the pin guide device 4 may be removed by pulling it over the guide pin, leaving the pin in place as shown in FIGS. 11A and 11B. In some embodiments, the surgical guide pin 23 enters only one surface of the bone and does not exit the bone at another surface (not shown). Additional tools such as a reamer may be used to shape the bone and/or an annulated drill to create a pilot hole for an implant. An implant may subsequently be passed into the pilot hole made by the drill. In some embodiments, the implant has an annular opening, and the implant is passed over the guide pin such that the guide pin fits into the annular opening of the implant. In some embodiments, the pin guide device 4 and placement tool 1 are used to guide an implant into the glenoid portion of the scapula bone.

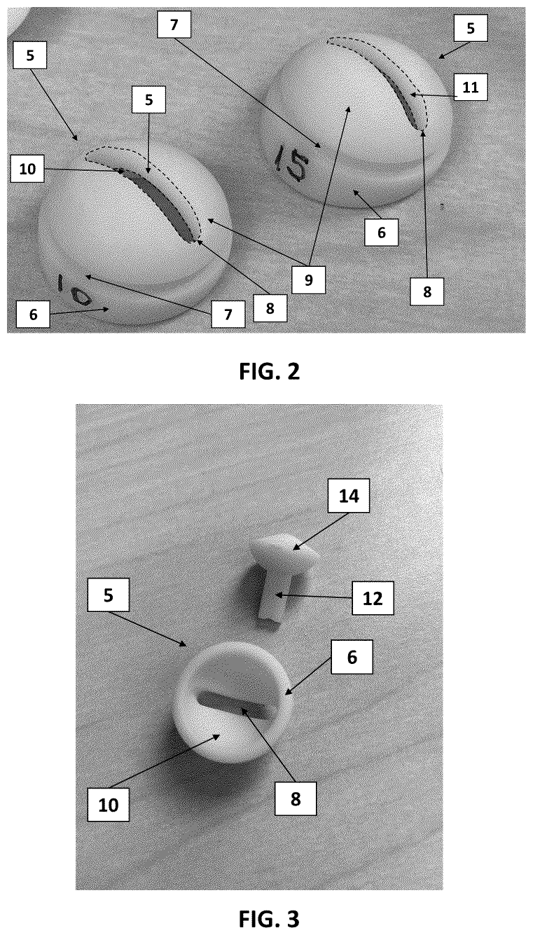

FIG. 2 shows a closeup of two example cup portions 5 of a pin guide device 4 at different angles. Cups 5 each have an outer convex surface 9, an inner concave surface 10 (best seen in FIG. 3), and a cup wall 11 between the outer convex surface 9 and inner concave surface 10. Cups 5 also comprise an elongated opening 8 extending through the cup wall 11. In some embodiments, an annular indentation 7 is disposed within the outer convex surface 9. System embodiments may be provided in kits comprising multiple cups 5 of different diameters, different relative angles between base 6 and elongated opening 8, and/or different angles of annular indentation 7 relative to base 6.

FIG. 3 depicts an embodiment of the pin guide device 4 where the cup 5 and the pin receiving member 14 are decoupled. The cup 5 has a substantially round base 6, an inner concave surface 10, and an elongated opening 8 disposed through the wall 11 of the cup 5. In some embodiments, the elongated opening 8 shape may be approximately oval, rectangular, or combinations thereof. In some embodiments, the length of the elongated opening 8 is longer than the width of the elongated opening 8. FIG. 3 also shows stem 12 protruding from the pin receiving member 14, In some embodiments, the pin receiving member comprises a rounded surface at the base of the stem 12 configured to contact the inner concave surface 10, as shown in FIG. 3.

FIG. 4 shows a pin guide device 4 where the cup 5 is coupled to the pin receiving member 14, with stem 12 protruding from elongated opening 8. In some embodiments, the stem 12 is configured to slide within the elongated opening 8 of cup 5. The elongated opening 8 may extend for up to 180 degrees within the wall 11 of cup 5. In some embodiments, the pin receiving member 14 is configured to lock at a predetermined location within the elongated opening 8 such that the stem 12 is held at a desired angle relative to base 6 to target a predetermined bone location for subsequent implant placement. The predetermined location may be determined prior to surgery based on the patient anatomy and a pre-operative plan and coincide with the ideal location for an implant. In some embodiments, the pin receiving member 14 is configured to be both adjustable and able to lock during a surgical procedure. In some embodiments, the cup 5 comprises one or more second mating members configured to interact with or connect with mating members on the pin receiving member 14 to hold stem 12 in a desired location.

Given to the rotation of the cup 5 with base 6 in contact with bone, and the sliding of stem 12 within the elongated opening 8 of the cup, a surgeon placing a guide pin using the pin guide device 4 is provided with a broad range of motion along an x-y coordinate system to ensure the proper location and orientation for guide pin placement. As such, embodiments described herein provide a system that can be configured from off the shelf components to create a customized angle of insertion specific to the needs of the patient.

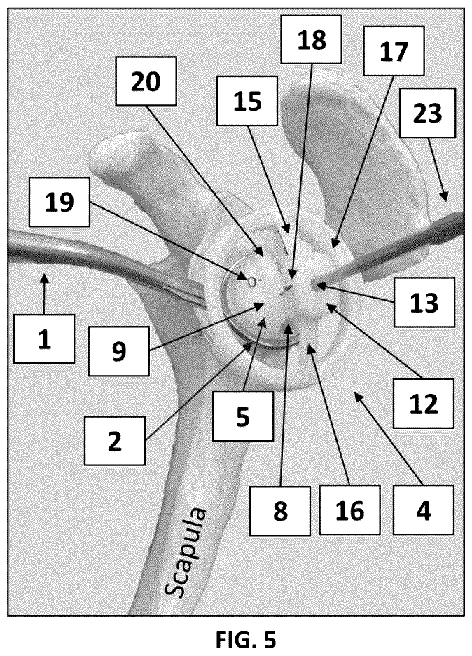

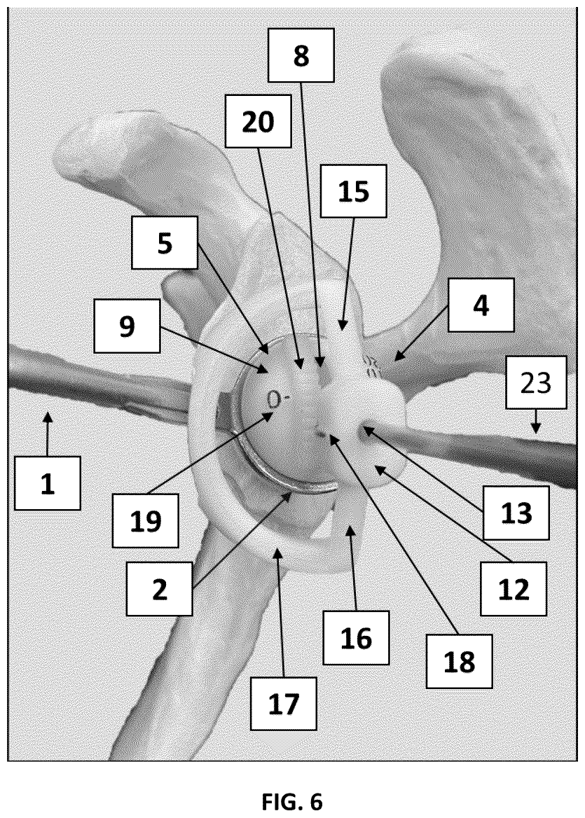

FIG. 5 shows a pin guide device 4 where the cup 5 is coupled to the pin receiving member 14, with stem 12 protruding from an elongated opening 8 within cup 5. In this embodiment, the stem comprises a first bridge 15 and a second bridge 16 which are oriented perpendicular to the longitudinal axis of the stem 12. The distal ends of the first bridge 15 and the second bridge 16 are connected by a substantially round implant sizer 17. The implant sizer 17 may have an opening between the stem and the implant sizer 17. As shown in FIG. 6, the implant sizer 17 may also be substantially semi-circular, or assume a different suitable configuration. In some embodiments, the implant sizer 17 is configured to match the diameter of a glenoid implant. A surgeon may use the implant sizer to visually assess the bone coverage the surgical implant location has for the implant. In embodiments where the implant sizer 17 is substantially semi-circular, the implant sizer 17 may be rotated around the surgical guide pin 23 providing a surgeon the ability to check the location of an implant while still having room to maneuver surgical equipment in the open substantially semi-circular area above the pin guide device 4 not occupied by the implant sizer 17.

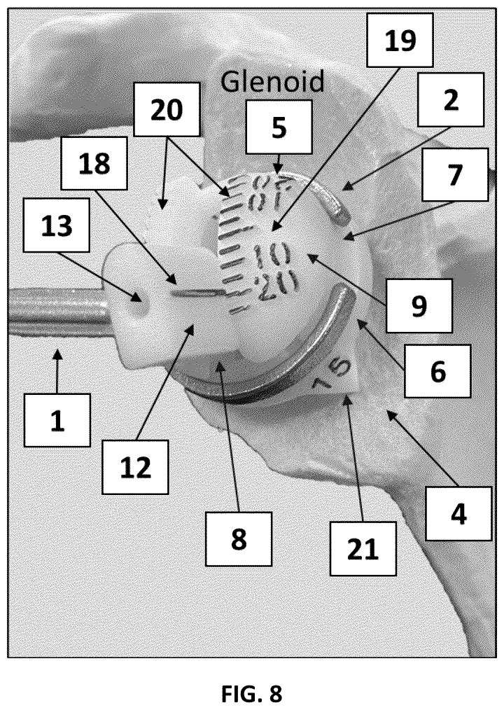

In some embodiments, the outer convex surface 9 comprises an outer convex mating surface 20 disposed on one or both sides of the elongated opening 8. The convex mating surface 20 may comprise one or more mating members in the form of ratchet teeth or other suitable structures, as shown in FIGS. 5-10. In some embodiments, the convex surface 9 further comprises offset angle markings 19 denoting the offset angle of a longitudinal axis of stem 12 relative to the cup base 6, as shown most clearly in FIGS. 8-10. In embodiments, the offset angle may range from zero to 90 degrees. Offset angle markings 19 may coincide with one or more ratchet teeth of the convex mating surface 20. As most clearly seen in FIGS. 8-10, the stem 12 may include a visible longitudinal axis marker 18 on an outer surface thereof, aligned with the longitudinal axis of the stem 12. In some embodiments, the longitudinal axis marker 18 may be raised to allow it to interface with the outer convex mating surface 20 to lock the stem 12 at a predetermined angle. In some embodiments, the longitudinal axis marker 18 will align with the offset angle markings 19 where the marking coincides with angle the longitudinal axis of the stem 12 forms relative to the base 6.

FIGS. 5 and 6 show the placement of pin guide device 4 on the glenoid portion of the scapula using a guide placement tool 1, where the arm 2 holds the pin guide device 4. A surgical guide pin 23, shown in FIG. 6, is disposed in the pin receiving member 14 through the passageway 13.

Embodiments may include protrusions extending from the base 6 of cups 5. For example, the embodiment shown in FIG. 7 includes a first protrusion 21, a second protrusion 22, and a third protrusion (not shown) extending from the bone facing surface of the base 6. The protrusions are of any suitable configuration and are triangular in the embodiment shown in FIG. 7 such that they are tapered from the base 6 to a point. The protrusions may be evenly or unevenly spaced along the base 6 to provide an even surface of the stem facing surface of the base 6. In embodiments, the one or more of the protrusions are sized differently from one or more of the other protrusions. For example, the first protrusion 21 may be longer than the other protrusions by up to about 20 millimeters. As can be seen in FIGS. 7, 8, and 10, the protrusions extending from the base 6 may include visible markings to indicate protrusion length or position information. In some embodiments, the visible markings indicate an offset length or additional protrusion length relative to the other protrusions.

In some embodiments, the base 6 is a separate component from the cup 5 as shown in FIG. 7. Such embodiments allow for the selection of a base having a desired offset to address patient-specific needs, such as the depth of a bone defect. For example, a protrusion extending from the base 6 having a suitable length may be placed in a bone defect during surgery to create a level surface of the stem facing surface of the base 6. For such embodiments, a cup 5 may be removably attached to base 6 such as by aligning or connecting a first alignment feature, such as protrusion extending from cup 5, with a second alignment feature, such as interfacing notch 24 on base 6, or by other suitable means.

FIGS. 5, 8, 9, and 10B show an embodiment where a first end of the elongated opening 8 extends through one edge of the cup 5. The elongated opening 8 may have a second end that does not extend to the edge of the cup. In some embodiments, the elongated opening 8 has a second end that extends through the edge of the cup opposite the first end. The cup 5 and elongated opening 8 may be configured to allow the stem 12 to slide into the elongated opening through the open end of the elongated opening 8. As best shown in FIG. 8 the base 6 may be configured to prevent the stem 12 from decoupling with the inner concave surface 10 of the cup 5.

In some embodiments, the pin guide device 4 and pin guide device placement tool 1 are made of a biocompatible material such as polymers, ceramics, metals, or combinations thereof. Any of the materials used to make surgical instruments known to those skilled in the art may be used to make the pin guide device 4 or pin guide device placement tool 1. In some embodiments, the material used to make the pin guide device 4 and pin guide device placement tool 1 are made of materials that can be sterilized. The sterilization process can be performed using any of the techniques known to one skilled in the art including, but not limited to, dry-heat sterilization, wet heat sterilization, radiation, ethylene oxide gas sterilization, hydrogen peroxide sterilization, bleach sterilization, hypochlorite sterilization, nitrogen dioxide sterilization, plasma sterilization, steam sterilization, autoclaving, boiling, or combinations thereof.

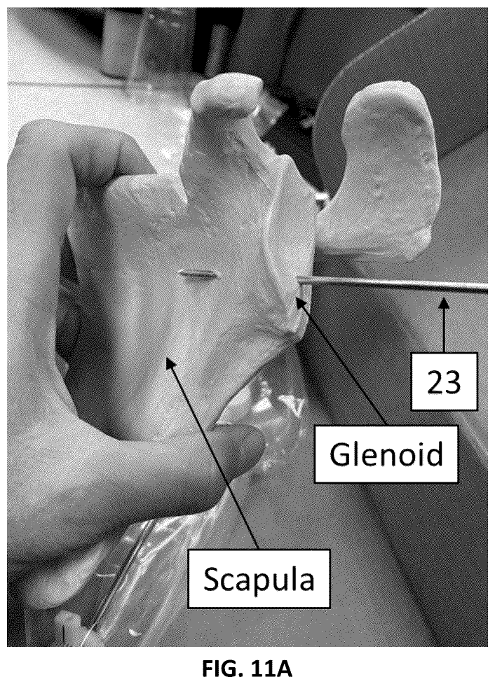

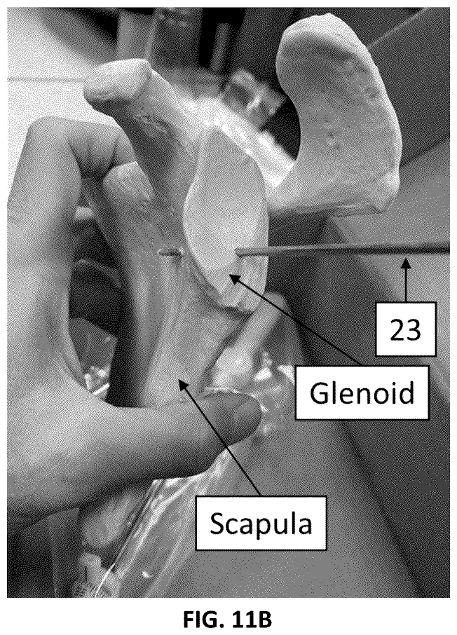

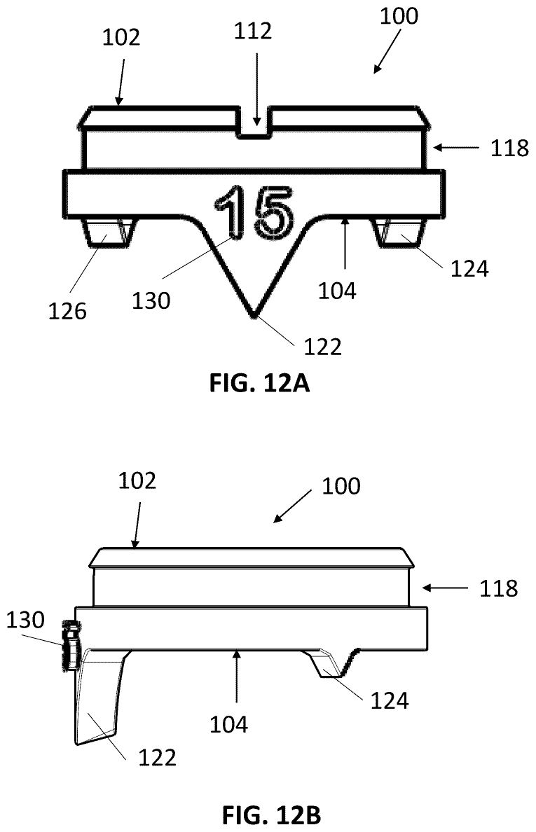

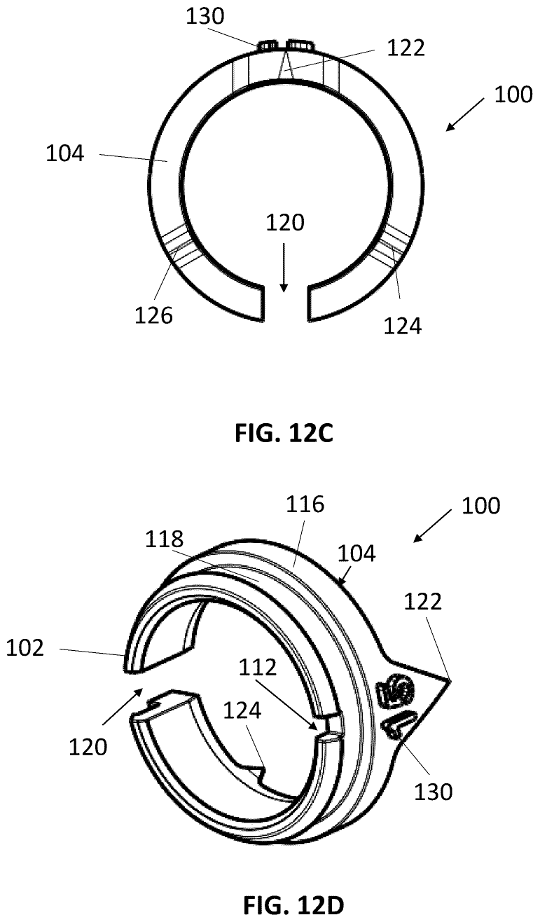

FIGS. 12A-12D show various views of a base 100 according to embodiments described herein. FIG. 12A illustrates a side view of a base 100 having a first surface 102, a second surface 104, an interfacing notch 112, an annular indentation 118, a first protrusion 122, a second protrusion 124, a third protrusion 126, and a protrusion marking 130. In some embodiments, the first protrusion 122, the second protrusion 124, and the third protrusion 126 extend from a bone facing surface of the base 100. In some embodiments, the first protrusion 122, the second protrusion 124, and the third protrusion 126 are tapered away from the bone facing or second surface 104 of the base 100. In some embodiments, the protrusion marking 130 is disposed on the outward facing surface of a protrusion. In some embodiments, the protrusion marking 130 is disposed on the outward facing surface of the first protrusion 122. In some embodiments, the protrusion marking 130 corresponds to the length of a protrusion. In some embodiments, the protrusion marking 130 corresponds to the length of first protrusion 122. In some embodiments, the protrusion marking 130 corresponds with an approximate depth of a defect in a patient's bone. In some embodiments, the first protrusion 122 corresponds with an approximate depth of a defect in a patient's bone.

In some embodiments, the annular indentation 118 is disposed radially about the longitudinal axis of the base 100 and into the outer surface of the base 100. In some embodiments, an interfacing notch 112 is disposed in the surface of the first surface 102. In some embodiments, the interfacing notch 112 is substantially rectangular in shape.

FIG. 12B illustrates a perspective side view of FIG. 12A according to embodiments described herein. As shown in FIG. 12B, for example, the first protrusion 122 may be longer than the second protrusion 124. In some embodiments, the first protrusion 122 is longer than either the second protrusion 124, the third protrusion 126, or both the second protrusion 124 and the third protrusion 126.

FIG. 12C illustrates a bottom view of FIG. 12A. As shown in FIG. 12C, for example, the first protrusion 122, the second protrusion 124, and the third protrusion 126 may be evenly distributed around the outer edge of the second surface. In some embodiments, the base 100 has a base bore extending through the longitudinal axis. As shown in FIGS. 12C and 12D, for example, a base gap 120 may extend from the outer surface of the base 100 through to the open base bore. FIG. 12D illustrates an angled top and side view of FIG. 12A according to embodiments described herein.

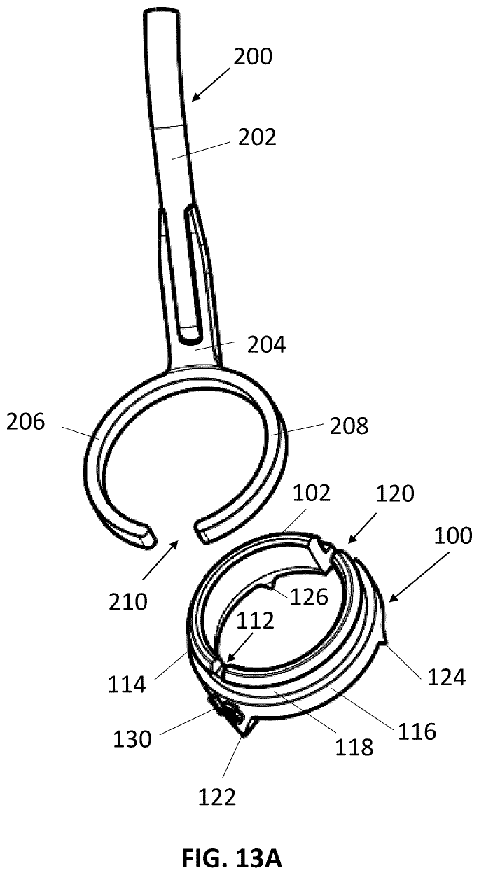

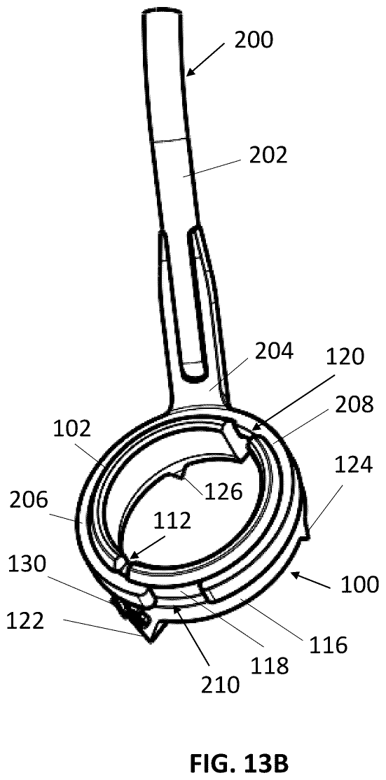

FIGS. 13A and 13B show a base 100 and a guide device placement tool 200 according to embodiments described herein. FIG. 13A illustrates an exploded view of the base 100 and the guide device placement tool 200 according to embodiments described herein. In some embodiments, the guide device placement tool 200 has a handle 202, an arm 204, a first hand 206, a second hand 208, and a placement tool gap 210. In some embodiments, the arm 204 extends from a distal end of the handle 202. In some embodiments, the first hand 206 and the second hand 208 extend from the distal end of the arm 204 at an angle approximately perpendicular to the arm 204. In some embodiments, the arm 204 extends from a distal end of the handle 202. In some embodiments, the first hand 206 and the second hand 208 extend from the distal end of the arm 204 radially. In some embodiments, the distal ends of the first hand 206 and the second hand 208 are separated by a placement tool gap 210.

FIG. 13B illustrates a coupled view of the base 100 and the guide device placement tool 200 according to embodiments described herein. In some embodiments, the first hand 206 and the second hand 208 are configured to move towards each other. In some embodiments, the first hand 206 and the second hand 208 are configured to mate with the annular indentation 118 of the base 100.

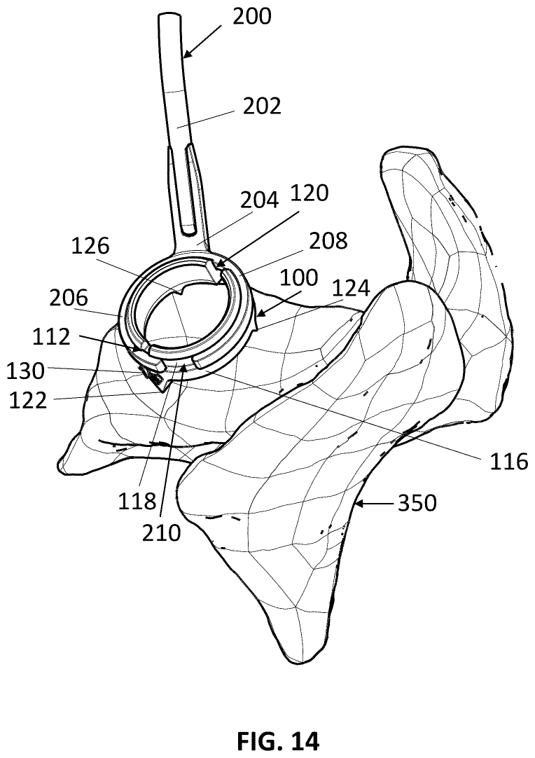

FIG. 14 shows a base 100 coupled with a guide device placement tool 200 placed over a glenoid portion of a scapula bone 350 according to embodiments described herein. The first protrusion 122, the second protrusion 124, and the third protrusion 126 may be configured to interface with the bone 350 and create a flat surface for the first surface 102 of the base 100.

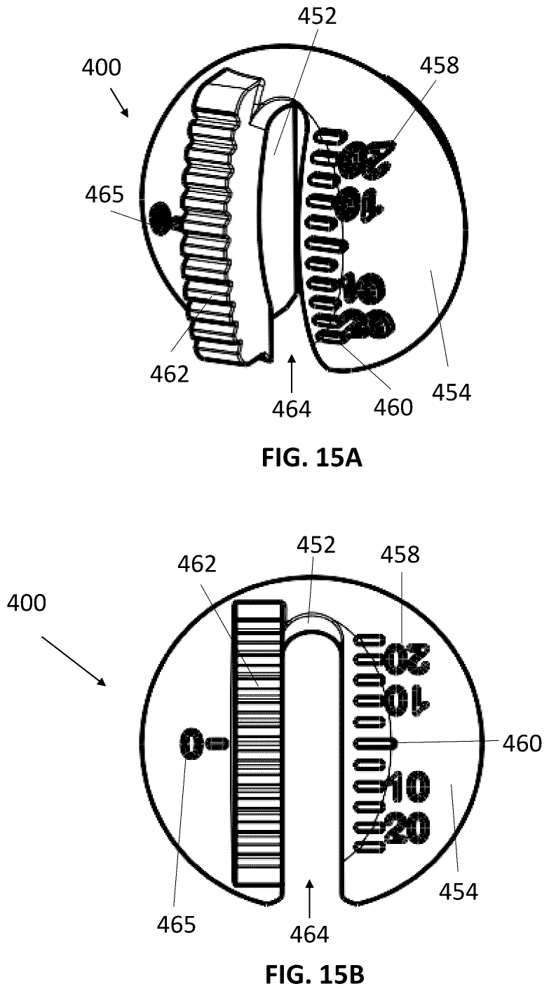

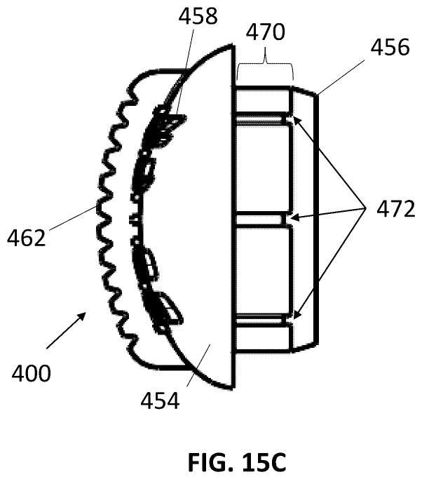

FIGS. 15A-15C show various views of cup 400. FIG. 15A illustrates an angled top view of a cup 400 having a cup wall 452, an outer convex surface 454, an offset angle numbering 458, an offset angle marking 460, an outer convex mating surface 462, an elongated opening 464, and a zero offset marker 465. In some embodiments, the cup wall 452 extends between the outer convex surface 454 and the bone facing cup surface 456. In some embodiments, the cup wall defines the interior surface of the elongated opening 464. The elongated opening 464 may extend from one end of the cup 400 at least partially though the outer convex surface 454. In some embodiments, the outer convex mating surface 462 extends from the outer convex surface 454. In some embodiments, the outer convex mating surface 462 comprises a plurality of teeth. In some embodiments, the outer convex mating surface 462 is disposed adjacent to the elongated opening 464. In some embodiments, an offset angle marking 460 is disposed adjacent to the elongated opening 464. In some embodiments, the outer convex mating surface 462 is disposed adjacent to the elongated opening 464 on a first side of the elongated opening 464 and the offset angle marking 460 is disposed adjacent to a second side of the elongated opening 464. In some embodiments, the offset angle marking 460 corresponds to an offset angle numbering 458. In some embodiments, an offset angle numbering 458 is aligned with at least one of the offset angle marking 460. In some embodiments, the base second surface 104 is configured to couple with the glenoid portion of the scapula 350.

FIG. 15B illustrates a top-down view of 15A. In some embodiments, the cup 400 has a zero offset marker 465 disposed on the outer convex surface 454 and aligned along a central axis of the cup 400.

FIG. 15C illustrates a side view of FIG. 15A according to embodiments described herein. As shown in FIG. 15C, cup 400 may extend from the outer convex surface 454 towards a bone facing cup surface 456. In some embodiments, the cup 400 has a fluted portion 470 disposed between the bone facing rim of the outer convex surface 454 and the bone facing cup surface 456. In some embodiments, the fluted section 470 may have one or more flutes 472 disposed at least partially in the outer surface and extending along the longitudinal axis of the cup 400. In some embodiments, the cup 400 is tapered from the fluted portion 470 to towards the bone facing cup surface 456.

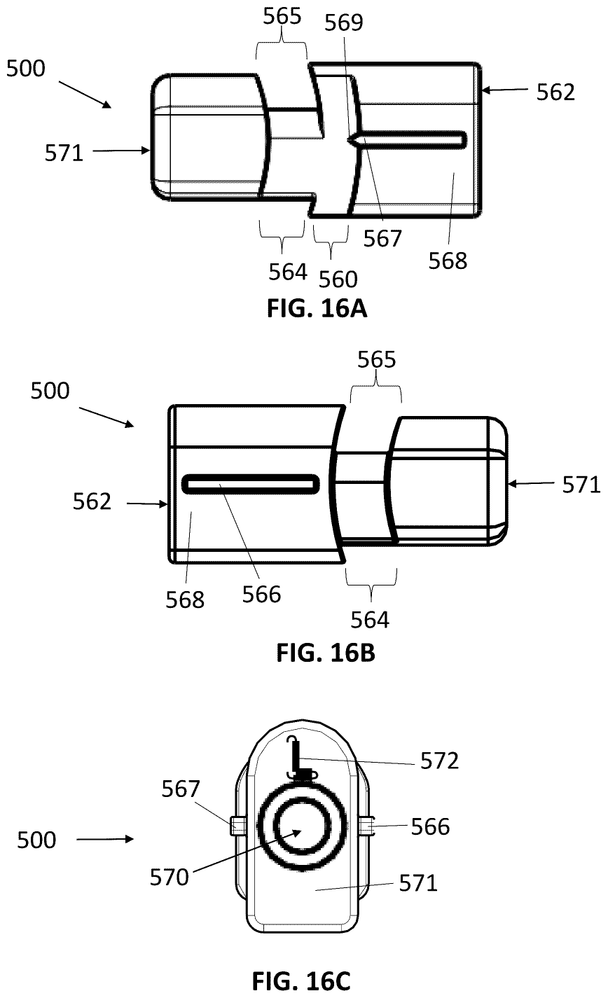

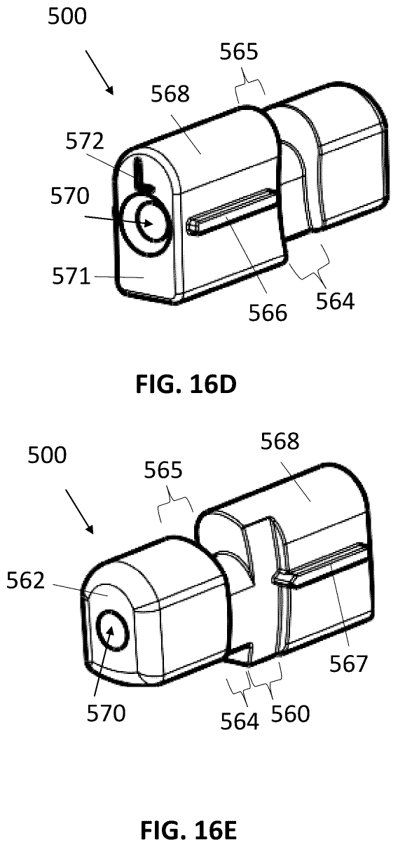

FIGS. 16A-16E show various views of a stem 500 according to embodiments described herein. FIG. 16A illustrates a first side view of the stem 500 having a proximal surface 571, a bone facing or distal surface 562, and a stem outer surface 568 therebetween.

In some embodiments, the stem has a first longitudinal axis marker 566 (see e.g., FIG. 16B) and a second longitudinal axis marker 567 disposed along the longitudinal axis of a stem outer surface 568. In some embodiments, a notch having a first notch portion 560, a second notch portion 564, and a third notch portion 565 may be disposed at least partially in the stem outer surface 568. In some embodiments, the first notch portion extends through about half of the stem outer surface. The first notch portion 560 may be configured to form a window at a proximal end of the second longitudinal axis marker 567. In some embodiments, the first notch portion is disposed between the combined second and third notch portions and the proximal end of the longitudinal axis marker 567. In some embodiments, the second and third notch portions are recessed radially inward from the stem outer surface 568. In some embodiments, the second notch portion 565 is recessed further towards the center of the stem than the third notch portion 564.

FIG. 16B illustrates a second side view of FIG. 16A, illustrating the stem 500 having the first longitudinal axis marker 566 extending from the outer stem surface along the longitudinal axis.

FIG. 16C illustrates a top view of the stem 500 illustrated FIG. 16A, according to embodiments described herein. As shown in FIG. 16C, for example, one or more of the first longitudinal axis marker 566 and the second longitudinal axis marker 567 may be raised from the stem outer surface 568. In some embodiments, such as illustrated in FIG. 16C, for example, the stem 500 has a generally “D” shape. In some embodiments, the stem 500 is generally rectangular in shape. In some embodiments, the stem 500 is generally circular, ellipsoid, triangular, square, pentagonal, hexagonal, heptagonal, octagonal, or any combination thereof. The stem 500 may have a stem bore extending from the distal stem surface 571 to the proximal stem surface 562. In some embodiments, the stem bore 570 is generally cylindrical in shape. In some embodiments, the stem bore 570 is configured to have a diameter larger than the diameter of a surgical guide pin.

In some embodiments, a proximal surface marker 572 is disposed on the proximal stem surface 571. The proximal surface marker 572 may be configured to indicate which surface of the stem is the proximal surface 571. The proximal surface marker 572 may have a generally “L” shape.

FIG. 16D illustrates an angled top and side view of the stem 500 illustrated in FIG. 16A according to embodiments described herein. In some embodiments, the stem bore 570 may be at least partially beveled from the distal or proximal stem surface towards the center of the stem.

FIG. 16E illustrates an angled bottom side view of FIG. 16A according to embodiments described herein. As shown in FIG. 16E, for example, the first notch portion may extend at least partially proximal to the tip of the second longitudinal axis marker 567. In some embodiments, the second longitudinal axis marker 567 is tapered towards the distal stem surface 562. In some embodiments, the distal end of the longitudinal marker 567 extends through the first notch portion 560 and past the outer stem surface 568 to a raised position.

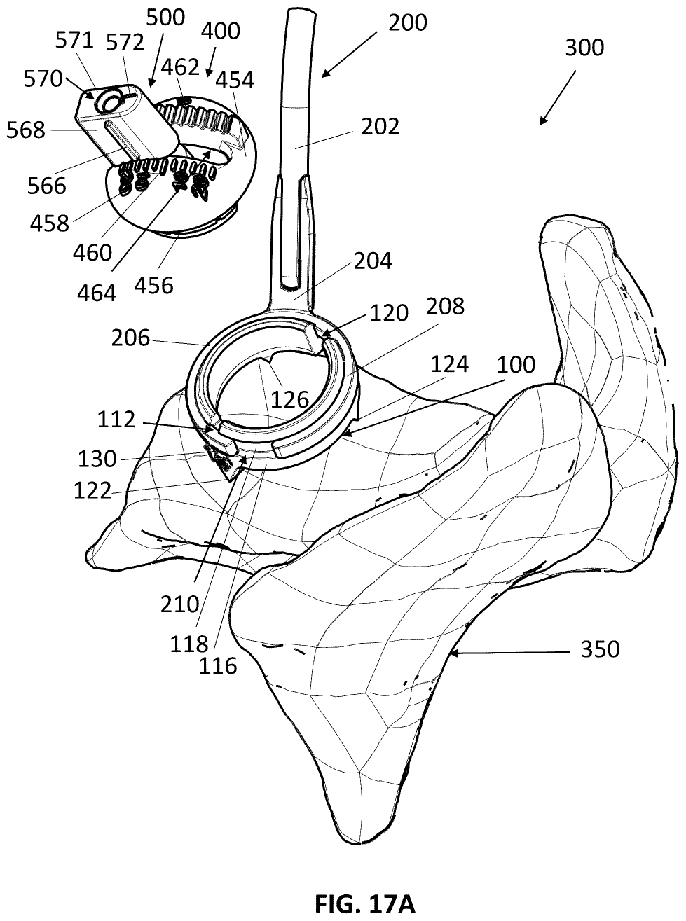

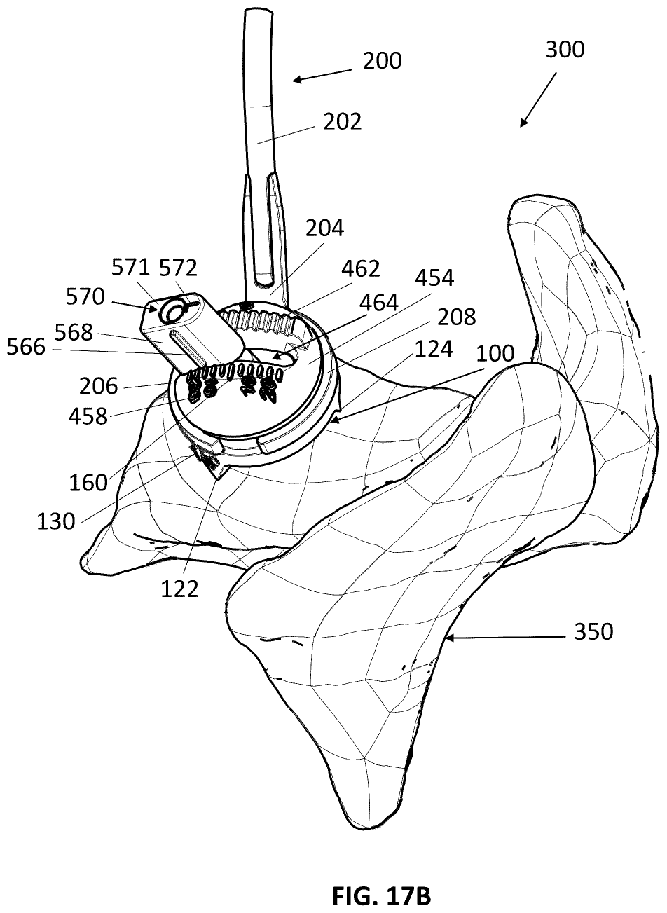

FIGS. 17A and 17B illustrate an exploded and compact view of a pin guide alignment system 300 according to embodiments described herein. FIG. 17A illustrates an exploded view of a pin guide alignment system 300 with a stem 500 coupled to the cup 400 and a guide placement tool 200 coupled to a base 100 and placed over a glenoid region of a scapula 350 according to embodiments described herein. In some embodiments, the stem 500 second notch portion 564 and third notch portion 565 are configured to couple with the interior surface or cup wall 452 of the elongated opening 464 of the cup 400. In some embodiments, the first longitudinal axis marker 566 is configured to align with an offset angle marking 460 to indicate the angle the stem bore 571 is relative to a zero-angle offset. In some embodiments, the bone facing cup surface 456 surface of the cup 400 is configured to couple with the base first surface 102. In some embodiments, the first protrusion 122 is located in a position corresponding to the largest defect in the scapula 350.

FIG. 17B illustrates a compact view of a pin guide alignment system 300 with a stem 500 coupled to cup 400 coupled to a base 100 and placed over a glenoid region of a scapula 350 by a guide placement tool 200 according to embodiments described herein. In some embodiments, the cup fluted portion 470 extending to the bone facing cup surface 456 is configured to fit at least partially within the open base bore. In some embodiments, the stem 500 and the cup 400 are one component. In some embodiments, the cup 400 and the base 100 are one component. In some embodiments, the stem 500, the cup 400, and the base 100 are one component. In some embodiments, the stem 500, the cup 400, and the base 100 are separate components. In some embodiments, the base 100 and the guide device placement tool 200 are one component.

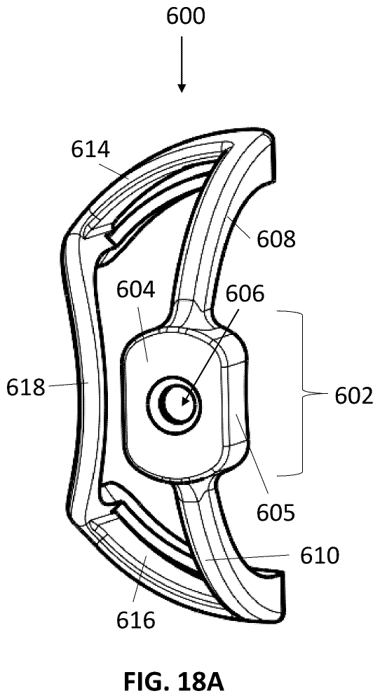

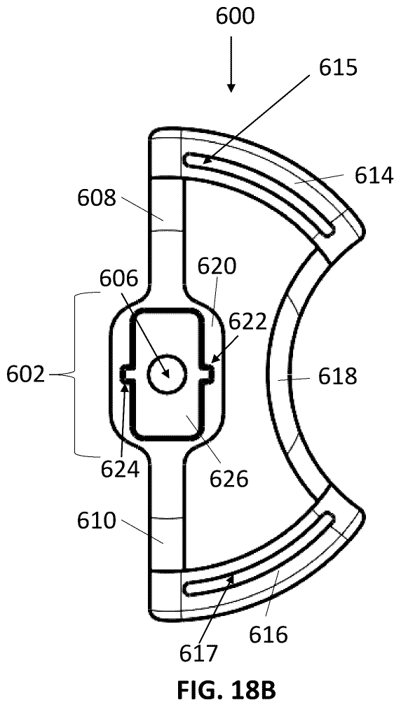

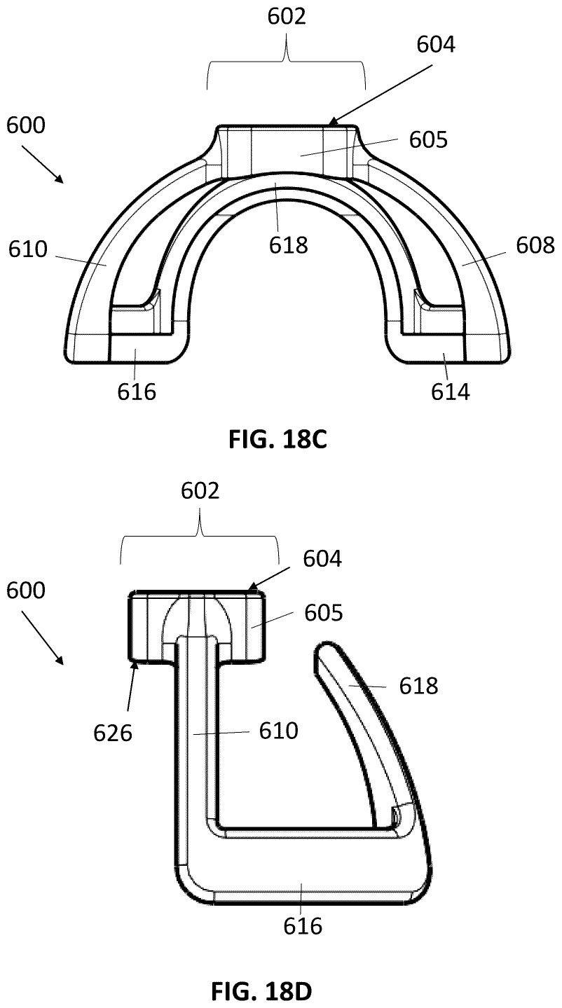

FIGS. 18A-18D illustrate various views of an implant sizer 600. FIG. 18A illustrates a top side angled view of an implant sizer 600 having an implant sizer platform 602, an implant sizer first bridge 608, an implant sizer second bridge 610, an implant sizer first wing 614, an implant sizer second wing 616, and an implant sizer arch 618. In some embodiments, the implant sizer platform 602 has an implant sizer platform proximal surface 604, an implant sizer platform distal surface 620, and an implant sizer platform wall 605 therebetween (see e.g., FIGS. 18C and 18D). In some embodiments, an implant sizer bore 606 is disposed at least partially through the implant sizer platform 602. In some embodiments, an implant sizer 600 has an implant sizer first bridge 608 and an implant sizer second bridge 610 extending from opposite ends of the implant sizer platform 602. In some embodiments, an implant sizer first wing 614 extends from the end of the implant sizer first bridge 608 furthest from the implant platform 602. In some embodiments, the implant sizer first wing 614 is at least partially curved. In some embodiments, an implant sizer second wing 616 extends from the end of the implant sizer second bridge 610 furthest from the implant platform 602. In some embodiments, the implant sizer second bridge 610 is at least partially curved. In some embodiments, the first and second wings 614 and 616 are at least partially curved towards each other. In some embodiments, the first and second wings 614 and 616 are connected by an arch 618. In some embodiments, the arch 618 extends from the first wing 614 to the second wing 616. In some embodiments, the arch 618 is curved from the ends of the first and second wings 614 and 616 towards the implant platform 602.

FIG. 18B illustrates a bottom view of the implant sizer 600 illustrated in FIG. 18A according to embodiments described herein. In some embodiments, the implant sizer 600 has an implant sizer recessed portion 626 disposed in the implant sizer platform distal surface 620. In some embodiments, the implant sizer bore 606 is centered in the implant sizer recessed portion 626. In some embodiments, the recessed portion has a first indentation 622 and a second indentation 624 on the opposite side of the recess as the first indentation 622. In some embodiments, the first indentation 622 and the second indentation 624 are configured to pair with the first longitudinal axis marker 566 and the second longitudinal axis marker 567, respectively. In some embodiments, a first lumen 615 is disposed at least partially through the first wing 614. In some embodiments, a second lumen 617 is at least partially disposed through the second wing 616. The first lumen 615 and second lumen 617 may convey flexibility to the implant sizer to allow a user to flex at least one of the first wing 614 and second wing 616 towards the cup 400. For example, a user may flex the first wing 614 and second wing 616 to determine placement of an implant relative to the bone. FIG. 18C illustrates a long side view of the implant sizer 600 illustrated in FIG. 18A according to embodiments described herein. FIG. 18D illustrates a short side view of the implant sizer 600 illustrated in FIG. 18A.

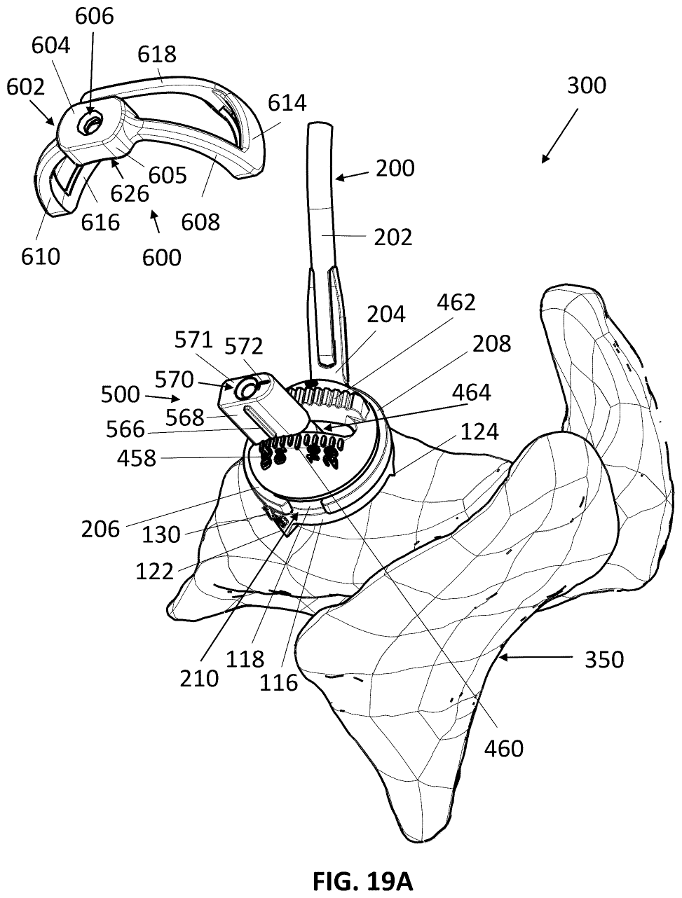

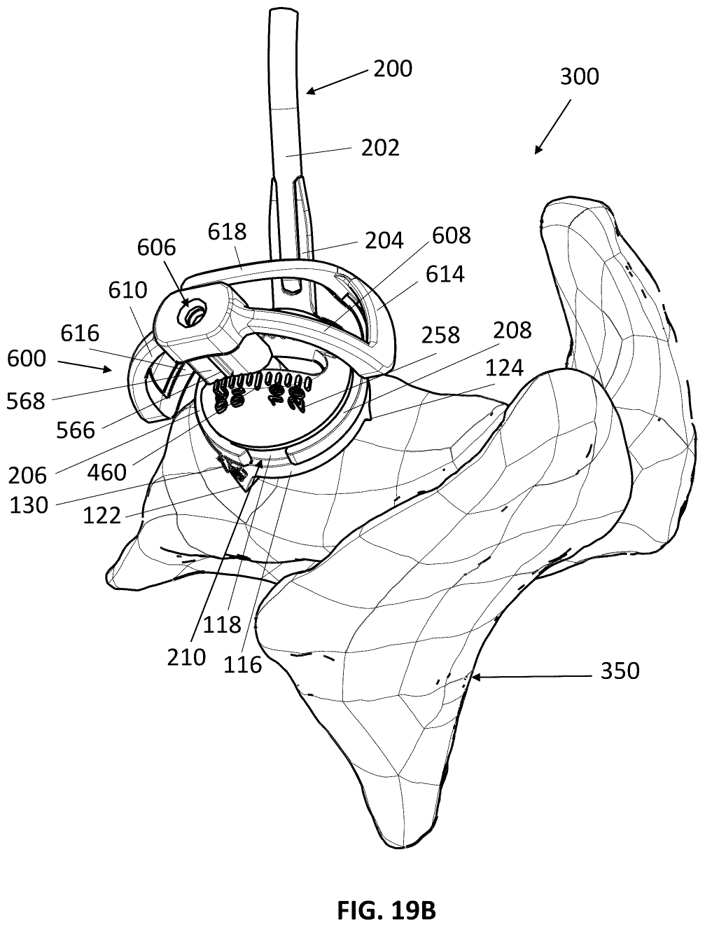

FIGS. 19A and 19B illustrate an exploded and compact view, respectively, of a pin guide alignment system 350 according to embodiments described herein. FIG. 19A illustrates an exploded view of a pin guide alignment system 350 with an implant sizer 600 configured to be coupled to a base 100 and placed over a glenoid region of a scapula bone 350 with a guide placement tool 200 according to embodiments described herein. FIG. 19B illustrates a compact view of a pin guide alignment system 300 with an implant sizer 600 coupled to a stem 500 coupled to cup 400 coupled to a base 100 and placed over a glenoid region of a scapula bone 350 by a guide placement tool 200 according to embodiments described herein. In some embodiments, the implant sizer recessed portion 626 is configured to mate with the stem 500. In some embodiments, the implant sizer bore 606 is configured to align with the stem bore 570. In some embodiments, the implant sizer bore 606 and stem bore 570 are configured to receive a surgical guide pin.

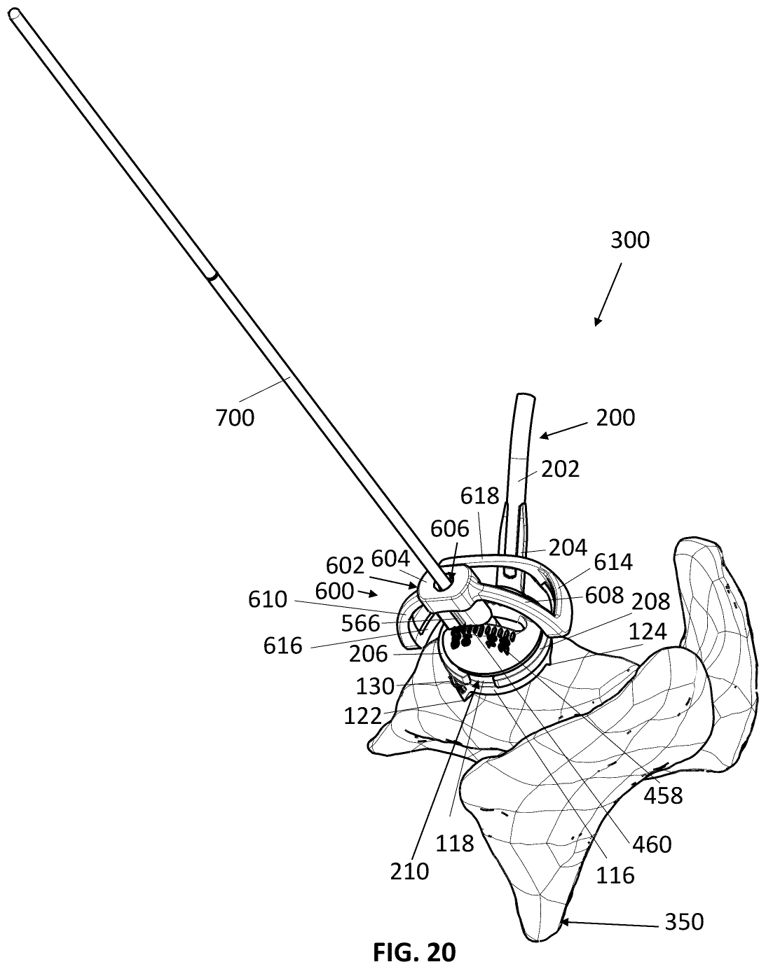

FIG. 20 illustrates a compact view of a pin guide alignment system 300 having a surgical guide pin 700 disposed through an implant sizer bore 606 coupled to a stem bore 570 coupled to cup 400 coupled to a base 100 and placed over a glenoid region of a scapula bone 350 by a guide placement tool 200 according to embodiments described herein.

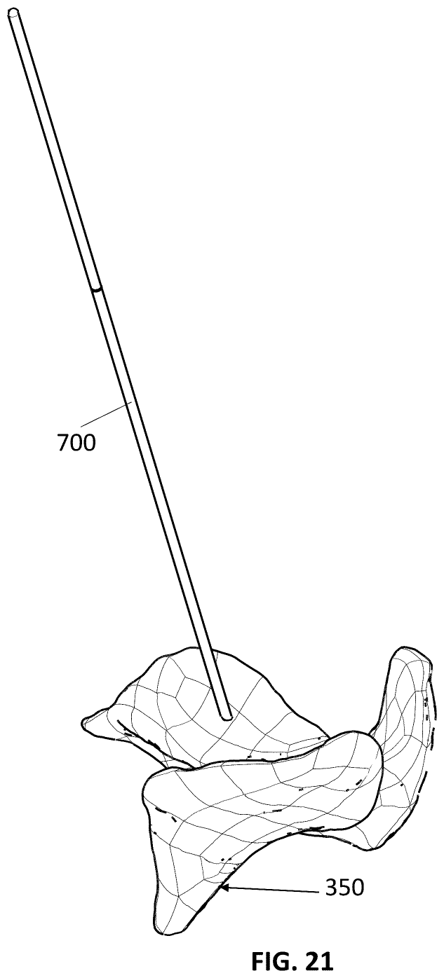

FIG. 21 illustrates a surgical guide pin disposed in a scapula bone 350 according to embodiments described herein. In some embodiments, the pin guide alignment system 300 is configured to be removed from the implant site by pulling it away from the bone 350 along the longitudinal axis of the surgical guide pin 700.

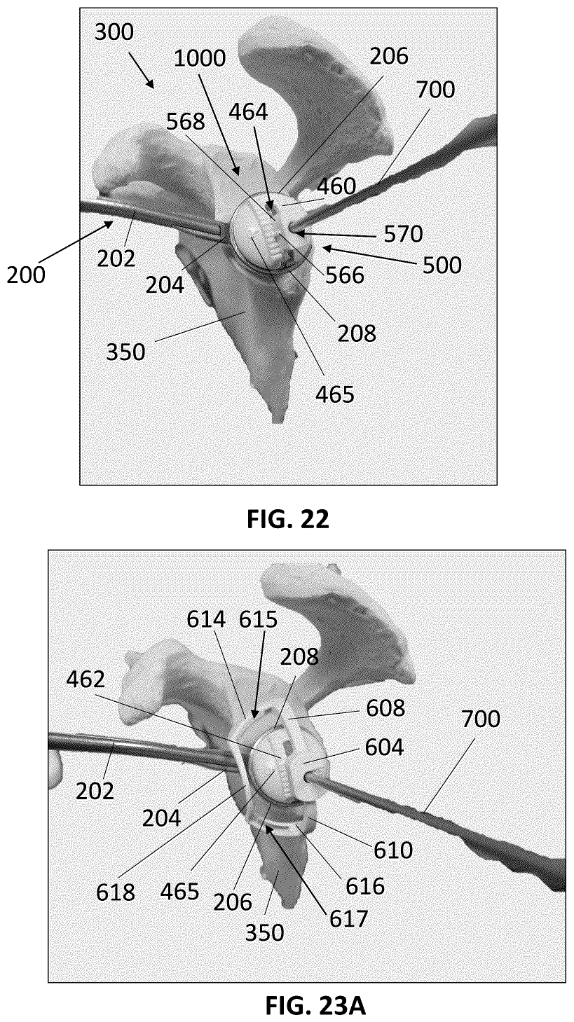

FIGS. 22-24 illustrate an example of a pin guide alignment system 300 placed over a glenoid region 1000 of a scapula bone 350 to insert a surgical guide pin 700. In some embodiments, a method to insert a surgical guide pin 700 includes coupling a base 100 with a cup 400. For example, the fluted portion 470 of the cup 400 may fit within the interior of the base 100. In some embodiments, the method includes coupling a stem 500 with the cup 400.

As shown in FIG. 22, in some embodiments, the method includes grasping the base 100 with a guide device placement tool 200 and placing the base 100 on top of the glenoid region 1000 of the scapula bone 350. In some embodiments, the base 100 is rotated until the first protrusion 122 fits within a defect of the glenoid 1000. In some embodiments, the stem 500 is moved to a predetermined offset position within the cup 400. In some embodiments, a surgical guide pin 700 is disposed through a stem bore and into the glenoid bone beneath.

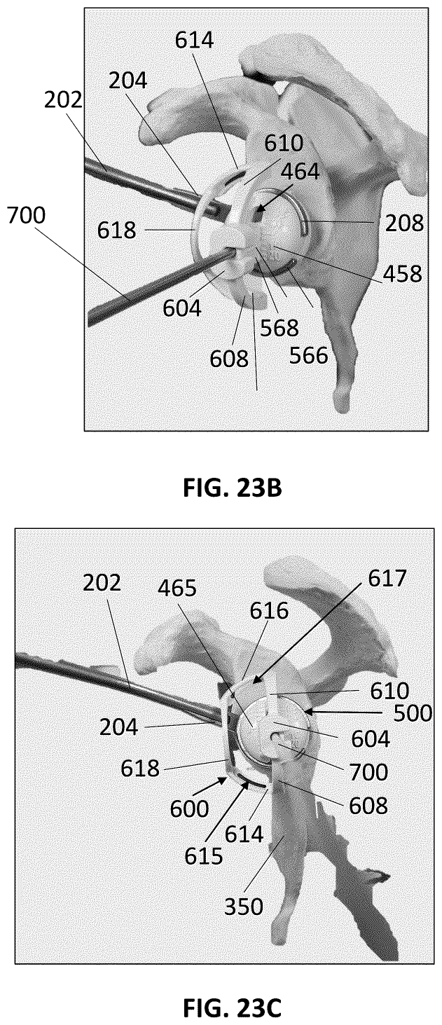

As shown in FIGS. 23A-23C, an implant sizer 600 may be placed over the surgical guide pin 700 to determine a fit of the implant size relative to the glenoid region 1000. In some embodiments, the method of placing and/or aligning a surgical guide pin further includes removing the implant sizer 600 and the pin guide alignment system 300 from the glenoid region 1000 by sliding it along the longitudinal axis of the surgical guide pin 700 away from the glenoid region 1000.

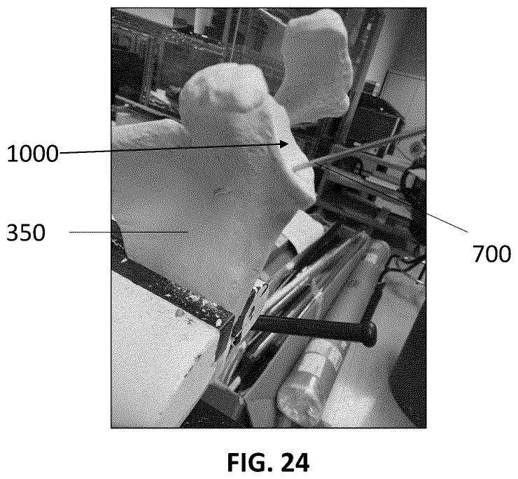

FIG. 24 illustrates a surgical guide pin 700 disposed in the glenoid region 1000 of a scapula bone 350 according to embodiments described herein. In some embodiments, the pin guide alignment system 300 (not shown) is configured to be removed from the implant site by pulling it away from the bone 350 along the longitudinal axis of the surgical guide pin 700.

Certain embodiments of the present invention were described above. It is, however, expressly noted that the present invention is not limited to those embodiments, but rather the intention is that additions and modifications to what was expressly described herein are also included within the scope of the invention. Moreover, it is to be understood that the features of the various embodiments described herein were not mutually exclusive and can exist in various combinations and permutations, even if such combinations or permutations were not made expressly herein, without departing from the spirit and scope of the invention. In fact, variations, modifications, and other implementations of what was described herein will occur to those of ordinary skill in the art without departing from the spirit and the scope of the invention. As such, the invention is not to be defined only by the preceding illustrative description.

1. A pin guide device comprising:
a cup comprising an inner concave surface, an outer convex surface, a cup wall between the inner and outer surfaces, and a substantially circular base;
an elongated opening extending through the cup wall; and
a stem comprising a passageway for receiving the pin, the stem extending through the elongated opening and slidable within the elongated opening.

2. The device of claim 1, wherein the stem protrudes from a pin receiving member comprising a rounded surface configured to contact the inner concave surface of the cup.

3. The device of claim 2, wherein the pin receiving member is configured to be locked at a desired location within the elongated opening.

4. The device of claim 2, wherein the cup comprises one or more first mating members, and the pin receiving member comprises one or more second mating members, wherein the first and second mating members are configured to interact with each other to hold the stem at a desired location within the elongated opening.

5. The device of claim 4, wherein the one or more first mating members are positioned on the outer convex surface of the cup.

6. The device of claim 1, further comprising one or more angle markings on the outer convex surface of the cup corresponding to an offset angle relative to the base.

7. The device of claim 6, further comprising an axis marking on the stem, the axis marking being parallel to a longitudinal axis of the stem and configured to be aligned with the one or more angle markings.

8. The device of claim 1, wherein the base is separable from the cup.

9. The device of claim 1, wherein the cup comprises a first alignment feature and the base comprises a second alignment feature, the first and second alignment features being connectable.

10. The device of claim 1, wherein the cup wall comprises a mating surface configured to receive a guide device placement tool.

11. The device of claim 10, wherein the mating surface is an annular indentation.

12. The device of claim 1, further comprising at least one protrusion extending from a bone facing surface of the base.

13. The device of claim 12, wherein the at least one protrusion comprises a first protrusion, a second protrusion, and a third protrusion.

14. The device of claim 13, wherein one of the first, second, and third protrusions is longer than the other protrusions.

15. The device of claim 1, further comprising a first bridge extending from the stem to a first end offset from the longitudinal axis of the stem, a second bridge extending from the stem to a second end offset from the longitudinal axis of the stem, and an implant sizer connecting the first end to the second end.

16. The device of claim 1, further comprising a first bridge extending to a first distal end perpendicular to the longitudinal axis of the stem, a second bridge extending to a second distal end perpendicular to the longitudinal axis of the stem, and an implant sizer connecting the first distal end to the second distal end.

17. The device of claim 16, wherein the implant sizer is circular or semi-circular.

18. (canceled)

19. (canceled)

20. (canceled)

21. (canceled)

22. (canceled)

23. A system for guiding a surgical pin into bone, comprising:
a pin guide device comprising:
a cup comprising an inner concave surface, an outer convex surface, a cup wall between the inner and outer surfaces, and a substantially circular base;
an elongated opening extending through the cup wall; and
a stem comprising a passageway for receiving the pin, the stem extending through the elongated opening and slidable within the elongated opening; and
a guide device placement tool comprising:
a handle, and
an arm configured to grasp the pin guide device.

24. The system of claim 23, wherein the cup wall comprises a mating surface configured to receive the guide device placement tool.

25. The device of claim 24, wherein the mating surface is an annular indentation.
